(19) United States
(12) Patent Application Publication
(10) Pub. No.: US 2021/0027864 A1
(43) Pub. Date: Jan. 28, 2021

(54) ACTIVE LEARNING MODEL VALIDATION

(71) Applicant: BENEVOLENTAI TECHNOLOGY LIMITED, London (GB)

(72) Inventors: Dean PLUMBLEY, London (GB); Marwin Hans Siegfried SEGLER, Southsea Hampshire (GB)

(73) Assignee: BENEVOLENTAI TECHNOLOGY LIMITED, London (GB)

(21) Appl. No.: 17/041,620

(22) PCT Filed: Mar. 29, 2019
(86) PCT No.: PCT/GB2019/050921

§ 371 Date: Sep. 25, 2020

(30) Foreign Application Priority Data
Mar. 29, 2018 (GB) 1805304.1

(51) Int. Cl. G16C 20/30; G16C 20/70

(52) U.S. Cl. CPC G16C 20/30; G16C 20/70

(57) ABSTRACT

Method(s), apparatus, and computer-implemented method(s) are provided for training a machine learning (ML) technique to generate a property model for predicting whether a compound has a particular property. An iterative procedure/feedback loop may be performed for generating the property model, the procedure including: generating a prediction result list for a plurality of compounds and their association with the particular property based on the property model; validating the property model based on compounds from the prediction result list having an association with the particular property; and updating the property model based on the property model validation. The procedure/loop may be repeated using the updated property model until it is determined the property model has been validly trained. The property model validation may include selecting a shortlist of compounds, performing simulation analysis and/or laboratory analysis on the shortlist of compounds in relation to the particular property and using the simulation and/or laboratory results in updating the property model.

The present application relates to apparatus, system(s) and method(s) for active learning and model validation.

### BACKGROUND

Informatics is the application of computer and informational techniques and resources for interpreting data in one or more academic and/or scientific fields. Cheminformatics' (a.k.a. chem(o)informatics) and bioinformatics includes the application of computer and informational techniques and resources for interpreting chemical and/or biological data. This may include solving and/or modelling processes and/or problems in the field(s) of chemistry and/or biology. For example, these computing and information techniques and resources may transform data into information, and subsequently information into knowledge for rapidly creating compounds and/or making improved decisions in, by way of example only but not limited to, the field of drug identification, discovery and optimization.

Machine learning techniques are computational methods that can be used to devise complex analytical models and algorithms that lend themselves to solving complex problems such as creation and prediction of whether compounds have one or more characteristics and/or property(ies). Although, there are a myriad of ML techniques that may be used or selected for predicting whether compounds have a particular property or characteristic, there is typically a shortage of training data for suitably training a ML technique to generate suitable a trained property model for predicting whether a compound has a particular property, which is referred to herein as a property model. If an ML technique is used to generate an property model based on insufficient labelled training data then the resulting property model may not be able to reliably predict whether a compound has a particular property for a broad range of compounds.

Generating a labelled training dataset for use in training an ML technique to generate accurate and reliable property models for predicting whether a compound has a particular property is costly, time consuming and error prone due to human error. The complexity of this task exponentially increases as the number of properties/characteristics that need to be predicted increases with each of a number of property models being used to predict whether a compound has one or more of the plurality of properties and/or characteristics. There is a desire to improve the training and use ML techniques for generating accurate and reliable property models for predicting whether compounds have one or more particular property(ies) to allow researchers, data scientists, engineers, and analysts to make rapid improvements in the field of drug identification, discovery and optimisation.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter; variants and alternative features which facilitate the working of the invention and/or serve to achieve a substantially similar technical effect should be considered as falling into the scope of the invention disclosed herein.

The present disclosure provides method(s) and apparatus for training a machine learning (ML) technique to generate a ML model for predicting whether a compound has a particular property (e.g. a property model). This uses an iterative procedure/feedback loop that may be performed for generating the ML model until it is considered to be validly trained. The procedure for each iteration of the feedback loop may include, by way of example only but is not limited to, generating a prediction result list for a plurality of compounds and their association with the particular property based on the ML model; validating the ML model based on compounds from the prediction result list having an association with the particular property; and updating the ML model based on the ML model validation. The procedure/loop may be repeated using the updated ML model until it is determined the ML model has been validly trained. As an example, the property model validation step may include selecting a shortlist of compounds, performing simulation analysis and/or laboratory analysis on the shortlist of compounds in relation to the particular property and using the simulation and/or laboratory results to update the ML model. The simulation and/or laboratory results may be used to form further labelled training data for training the ML technique to generate the updated ML model.

In a first aspect, the present disclosure provides a computer-implemented method for generating a ML model, also referred to herein as a property model, for predicting whether a compound has a particular property. The method comprising: training a ML technique to generate the property model; generating a prediction result list for a plurality of compounds and their association with the particular property using the property model; validating the property model based on compounds from the prediction result list having an association with the particular property; updating the property model based on the property model validation.

Preferably, the method including repeating at least the generating and validation step using the updated property model until determining the property model has been validly trained. The steps of generating, validating and updating may be part of a feedback loop, that may be repeated or iterated using the updated property model of the previous iteration until it is determined the property model has been validly trained and/or a suitable stopping criterion (e.g. maximum number of iterations, plateau in property model score, a peak in property model score, and the like etc.) has been met or reached.

Preferably, the method further includes generating a prediction result for a plurality of compounds and their association with the particular property using the property model; and validating the property model based on the compounds from the prediction result list having an association with the particular property.

Preferably, the ML technique is initially trained based on a labelled training dataset associated with a subset of the plurality of compounds in relation to the particular property. The subset of the plurality of compounds, may be a subset of the plurality of compounds used to generate the prediction result list.

Preferably, validating the property model further comprises validating a shortlist of compounds from the prediction result list having an association with the particular property; and updating the property model further comprises updating the property model based on training the ML technique with a labelled training dataset including the validated shortlist of compounds.

Preferably, updating the property model further comprising: generating a further labelled training dataset based on the validated shortlist of compounds and any previously labelled training dataset associated with the particular property; and retraining the ML technique based on the generated labelled training dataset.

Preferably, validating the shortlist of compounds further comprises: determining whether to perform laboratory experimentation based on the particular property and the shortlist of compounds; and in response to determining to perform laboratory experimentation, using experimental results from the laboratory experimentation to estimate the association each compound on the shortlist of compounds has with the particular property.

Preferably, determining to perform laboratory experimentation is based on one or more from the group of: a number of validation iterations exceeding a validation iteration threshold in which simulation analysis has been consecutively performed for validating the shortlist; an indication that laboratory analysis will yield an improvement in an ML score for the property model based on previous property model scores calculated from corresponding prediction result lists generated after each shortlist of compounds has been validated; or a combination on a number of validation iterations and an indication that laboratory experimentation will provide an improved property model.

Preferably, determining whether to perform laboratory experiments further comprises: determining whether the selected shortlist of compounds has substantially changed from a previously selected shortlist of compounds; in response to determining that the selected shortlist of compounds has not substantially changed from the previously selected shortlist of compounds, electing to perform laboratory experimentation on a selected subset of compounds from the selected shortlist of compounds.

Preferably, validating the shortlist further comprises: determining whether to perform simulation analysis (or computer simulation analysis) based on the particular property and the shortlist of compounds; and in response to determining to perform simulation analysis, using simulation results from the simulation analysis to estimate the association each compound on the shortlist of compounds has with the particular property.

Preferably, determining to perform simulation analysis or computer simulation/analysis is based on one or more from the group of: a number of validation iterations exceeding a validation iteration threshold in which simulation analysis has been consecutively performed for validating the shortlist; an indication that simulation analysis or computer simulation/analysis will yield an improvement in an ML score for the property model based on previous property model scores calculated from corresponding prediction result lists generated after each shortlist of compounds has been validated; or a combination on a number of validation iterations and an indication that simulation analysis will provide an improved property model.

Preferably, the number of validation iterations in which simulation analysis is performed consecutively is greater than the number of validation iterations in which laboratory analysis is performed.

Preferably, laboratory analysis is performed once for each of a plurality of generation and validation iterations in which simulation analysis is performed consecutively.

Preferably, the prediction result list comprises a prediction score of whether said each compound has the particular property, the method further comprising selecting the shortlist of compounds from the prediction result list based, at least in part, on the prediction score.

Preferably, validating the shortlist of compounds further comprises selecting one or more compounds for the shortlist of compounds from the prediction result list based on whether a compound has a prediction score indicative of a borderline prediction score.

Preferably, the prediction score comprises a certainty score, wherein compounds that are known to have the particular property are given a positive certainty score, compounds that are known not to have the particular property are given a negative certainty score, and other compounds are given an uncertainty score between the positive certainty score and negative certainty score.

Preferably, the certainty score is a percentage certainty score, wherein the positive certainty score is 100%, the negative certainty score is 0%, and the uncertainty score is between the positive and negative certainty scores.

Preferably, selecting the shortlist of compounds from the prediction result list further comprises selecting one or more compounds having an uncertain prediction result.

Preferably, selecting the shortlist of compounds from the prediction result list further comprises selecting one or more compounds that are dissimilar to the compounds used in any labelled training data used so far.

Preferably, selecting the shortlist of compounds from the prediction result list further comprises using a selection model for selecting the shortlist of compounds from the prediction result list, wherein the selection model is generated by training a reinforcement learning, RL, technique.

Preferably, generating the selection model based on the RL technique further comprising: selecting, using the selection model, a set of compounds for the shortlist of compounds from the prediction result list for validation; validating whether the selected shortlist of compounds has the particular property; and updating the property model based on the ML technique and the validated shortlist of compounds; generating an ML score and further prediction result list based on the updated property model; and determining whether to retrain the selection model to select a set of compounds for the shortlist of compounds based on the ML score and previous ML score(s).

Preferably, in response to determining to retrain the selection model, the method further comprising: reverting the updated property model to a previous property model when the ML score does not reach a property model performance threshold compared with the corresponding previous ML score; retaining or keeping the updated property model when the ML score is indicative of meeting or exceeding the property model performance threshold compared with the corresponding previous ML score; and retraining the selection model to select a set of compounds from the corresponding prediction result list based on the ML score; and repeating the generating the selection model steps including at least the steps of selecting, validating and updating the property model until the selection model is determined to be trained.

Preferably, determining the selection model is trained further comprises: comparing the retained/kept property model score with previous retained property model score(s); and determining the selection model has been validly trained based on a plateau of property model scores.

Preferably, determining whether the property model has been validly trained further comprises determining the property model has been validly trained based on an indication that further validation of a shortlist is unnecessary. Alternatively or additionally, preferably, determining the property model is validly trained further comprises: comparing a retained/kept property model score with previous retained property model score(s); and determining the property model has been validly trained based on a plateau of property model scores.

Preferably, validating the property model further comprising: generating a property model score based on the prediction result list; determining whether the property model has been validly trained based on the property model score and previous property model scores.

Preferably, determining whether the property model has been validly trained includes determining the property model has been validly trained based on a plateau of property model scores.

Preferably, the ML technique comprises at least one ML technique or combination of ML technique(s) from the group of: a recurrent neural network configured for predicting, starting from a first compound, a second compound exhibiting a set of desired property(ies); convolutional neural network configured for predicting, starting from a first compound, a second compound exhibiting a set of desired property(ies); reinforcement learning algorithm configured for predicting, starting from a first compound, a second compound exhibiting a set of desired property(ies); and any neural network structure configured for predicting, starting from a first compound, a second compound exhibiting a set of desired property(ies).

Preferably, the particular property includes a property or characteristic indicative of: a compound docking with another compound to form a stable complex; a ligand docking with a target protein, wherein the compound is the ligand; a compound docking or binding with one or more target proteins; a compound having a particular solubility or range of solubilities; a compound having a particular toxicity; any other property or characteristic associated with a compound that can be simulated based on computer simulation(s) and physical movements of atoms and molecules; any other property or characteristic associated with a compound that can be determined from an expert knowledgebase; and any other property or characteristic associated with a compound that can be determined from an experimentation. The particular property may further include a property, characteristic and/or trait indicative of: partial coefficient (e.g. LogP), distribution coefficient (e.g. LogD), solubility, toxicity, drug-target interaction, drug-drug interaction, off-target drug effects, cell penetration, tissue penetration, metabolism, bioavailability, excretion, absorption, drug-protein binding, drug-lipid interaction, drug-Deoxyribonucleic acid (DNA)/Ribonucleic acid (RNA) interaction, metabolite prediction, tissue distribution and/or any other suitable property, characteristic and/or trait in relation to a compound.

Preferably, the method of generating the property model may be repeated until it is determined the property model has been validly trained. Additionally, the method may include further training the property model by iterating over the steps of generating, validating and updating the property model until it is determined the property model has been validly trained or when a stopping criterion has been reached or met, wherein an updated property model from a previous or current iteration is used when repeating at least the generating, validating and updating steps in the next iteration.

In a second aspect, the present disclosure provides an apparatus comprising a processor, a memory unit and a communication interface, wherein the processor is connected to the memory unit and the communication interface, wherein the processor and memory are configured to implement the computer implemented method according to the first aspect, modifications thereof and/or as described herein.

In a third aspect, the present disclosure provides a ML model comprising data representative of a ML model generated by training a ML technique according to the computer-implemented invention of the first aspect, modifications thereof and/or as described herein.

In a fourth aspect, the present disclosure provides property model obtained or obtainable by the computer-implemented method according to the first aspect, modifications thereof and/or as described herein.

In a fifth aspect, the present disclosure provides an apparatus comprising a processor, a memory unit and a communication interface, wherein the processor is connected to the memory unit and the communication interface, wherein the processor and memory are configured to implement a ML model according to the third or fourth aspects and/or as described herein.

In a sixth aspect, the present disclosure provides a computer readable medium comprising data or instruction code representative of a ML model generated based on training a ML technique according to the computer implemented method of the first aspect, modifications thereof, and/or as described herein, which when executed on a processor, causes the processor to implement the ML model.

In a seventh aspect, the present disclosure provides a computer readable medium comprising data or instruction code representative of a ML model according to the third or fourth aspects and/or as described herein, which when executed on a processor, causes the processor to implement the ML model.

In an eighth aspect, the present disclosure provides a method for predicting whether a compound has a particular property using a ML model trained by the computer-implemented method according to the computer implemented method of the first aspect, modifications thereof, and/or as herein described.

In a ninth aspect, the present disclosure provides a system for generating a ML model (e.g. a property model) for predicting whether a compound is associated with a particular property, the system comprising: a model generation module for training a ML technique to generate the ML model; a model test module for generating a prediction result for a compound and their association with the particular property using the ML model; a validation module for validating the ML model based on the compound from the prediction result having an association with the particular property; and a model update module for updating the ML model based on the ML model validation.

Preferably, the system further includes one or more features of the first aspect, modifications thereof, or as described herein. Preferably, the model generation module, model test module, validation module, and/or model update module may be configured to implement the computer-implemented method of the first aspect, modifications thereof, and/or as described herein and the like. Preferably, the model generation module, model test module, validation module, and/or model update module may be further configured to implement one or more function or functionalities of one or more of the second to eighth aspects, modifications thereof, and/or as described herein and the like.

The methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1a is a flow diagram illustrating an example process for training a ML technique to generate and validate a property model to predict whether compounds have a particular property according to the invention;

FIG. 1b is a schematic diagram illustrating an example apparatus for implementing the example process of FIG. 1a according to the invention;

FIG. 2 is a table illustrating an example prediction result list output from a property model for a plurality of compounds according to the invention;

FIG. 3 is a schematic diagram illustrating an example apparatus for validating an property model according to the invention;

FIG. 4 is a schematic diagram illustrating an example apparatus for validating a shortlist of compounds for use in training a ML technique to generate a property model according to the invention;

FIG. 5 is a flow diagram illustrating an example process for selecting a shortlist of compounds for use in FIGS. 4a and 4b according to the invention; and

FIG. 6 is a schematic diagram of a computing device according to the invention.

Common reference numerals are used throughout the figures to indicate similar features.

### DETAILED DESCRIPTION

Embodiments of the present invention are described below by way of example only. These examples represent the best mode of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

The inventors have advantageously developed a method/mechanism that judiciously uses a combination of simulations and/or laboratory experiments on selected compounds in an iterative and semi-automated/automated approach that enhances the training of machine learning (ML) techniques for generating accurate and reliable ML models, e.g. ML models such as, by way of example only but not limited to, property models for predicting whether a compound exhibits or has a particular property. This mechanism may be particularly applicable when there is insufficient labelled training data for training the ML technique to generate, by way of example only but not limited to, an property model for predicting whether a compound has a particular property. The mechanism can enhance the labelled training dataset by selecting the best subset of compounds that should maximise or at least improve the performance of the property model whilst determining when to best validate the subset against the particular property via computer simulation or via laboratory experimentation. The property model can be updated based on the enhanced labelled training dataset. Thereafter, the mechanism may iteratively further enhance the labelled training dataset using another selected subset of compounds using primarily simulation, and when necessary, requesting and having laboratory experimentation performed on the minimum number of compounds or a subset of compounds that will enhance the performance of the property model.

Although the following description of the invention refers to, by way of example only but is not limited to, property models and/or ML models for predicting whether one or more compound(s) is associated or has a particular property (e.g. whether one or more entities is associated with a relationship), it will be appreciated by the skilled person that the present invention may be applied to other ML models for predicting whether an entity or input data has a particular relationship with another entity, or for classifying one or more entities and/or input data according to a particular relationship etc. The entities may include one or more compounds, drugs, proteins/genes or other biological entity and the like.

A predictive property model (or ML model for predicting whether a compound exhibits or has a particular property) can be configured to receive a compound as input and output data representative of a prediction for whether or not that compound has a particular property. For example, the property model may be configured to, by way of example only but is not limited to, predict whether a compound will bind to a particular protein; or predict whether the compound is soluble in water; or predict whether the compound is toxic to the human body or part of the human body; or predict any other property of interest in relation to compounds. However, the labelled training dataset may only contain data related to a few hundreds to a few thousand compounds in relation to the particular property. This is not enough data to properly train a ML technique to generate a property model that would predict whether a compound exhibits and/or has the particular property.

The quality of the property model may be improved by increasing the size of the labelled training dataset. For example, a plurality of compounds with an unknown association with the particular property may be tested in a laboratory via experimentation to measure whether or not they exhibit or are associated with the particular property. However, this is extremely costly for all but a few compounds. The inventors have developed a technique for limiting the number of compounds that are necessary to test in the laboratory whilst improving on the property model quality. This can be achieved by initially selecting a shortlist of compounds from a prediction result list of a plurality of compounds output from the property model. The shortlist is typically greater than the number of compounds that are usually sent for testing in a laboratory. Computer simulations based on molecular dynamics/interactions are used to validate the shortlist of compounds in relation to the particular property. The validation results from the computer simulations of the shortlist are fed back into the property model (e.g. using them to enhance the labelled training dataset and retraining the property model accordingly), which may output another prediction result list based on the plurality of compounds. Another shortlist may be selected, validated by computer simulation and fed back into the property model. These steps may be repeated until it is determined that laboratory testing will further enhance the quality of the property model. After laboratory testing, the laboratory results of the validated shortlist of compounds may be fed back into the property model (e.g. the laboratory results are used to further enhance the labelled training dataset and retrain the property model accordingly). The steps may be repeated with further simulation loops and/or laboratory experiment loops until it is considered the property model has been suitably trained.

Laboratory testing may be determined based on, by way of example only but not limited to, one or more of: determining that the simulation testing technique has been exhausted e.g. little or no improvement in the property model is being seen based on the simulations; it is observed that a very small shortlist of uncertain compounds is being output by the prediction result list; a maximum number of iterations using simulation for validating the shortlist has been reached; a minimum number of compounds have been selected for laboratory testing and it is determined these selected compounds should get a maximum number of improvements in the quality of the property model; and/or the overall property model performance score(s) of the property model plateaus compared with previous property model performance scores; or the property model performance score(s) is worse than previous property model performance scores, in which case, the property model is reverted to the best performing property model and a shortlist selected for laboratory experimentation; any other condition or criterion that may assist in enhancing the quality of the property model; and/or any combination of thereof.

The compounds may be selected for the shortlist of compounds for simulation and/or laboratory testing based on, by way of example only but is not limited to, one or more of: selecting those compounds that are most dissimilar to compounds already in the labelled training dataset; selecting those compounds that the property model is the least uncertain about regardless of whether those compounds exhibit the particular property or not (e.g. borderline cases); selecting those compounds using a ML selection model that has been trained for selecting the best compounds that result in improved ML quality; and/or any other combination thereof.

For example, the particular property may be related to docking, and the property model may be generated for predicting where a compound binds to a particular point or binding site. A compound in the selected shortlist for validation may be input to a computer docking simulation configured in relation to the binding site, which simulates whether or not the compound sticks/docks to the binding site e.g. a compound docking to a protein. The computer simulation may output validation results such as, by way of example only but not limited to, a docking score or data representative of how well the compound docked with the binding site. These results are fed back into the property model by using the output validation results to enhance the labelled training data and retrain the ML technique using the labelled training data to generate an updated property model (e.g. retrained property model).

A compound (also referred to as one or more molecules) may comprise or represent a chemical or biological substance composed of one or more molecules (or molecular entities), which are composed of atoms from one or more chemical element(s) (or more than one chemical element) held together by chemical bonds. Example compounds as used herein may include, by way of example only but are not limited to, molecules held together by covalent bonds, ionic compounds held together by ionic bonds, intermetallic compounds held together by metallic bonds, certain complexes held together by coordinate covalent bonds, drug compounds, biological compounds, biomolecules, biochemistry compounds, one or more proteins or protein compounds, one or more amino acids, lipids or lipid compounds, carbohydrates or complex carbohydrates, nucleic acids, deoxyribonucleic acid (DNA), DNA molecules, ribonucleic acid (RNA), RNA molecules, and/or any other organisation or structure of molecules or molecular entities composed of atoms from one or more chemical element(s) and combinations thereof.

Each compound has or exhibits one or more property(ies), characteristic(s) or trait(s) or combinations there of that may determine the usefulness of the compound for a given application. The property of a compound or property of interest may comprise or represent data representative or indicative of a particular behaviour/characteristic/trait of a compound when the compound undergoes a reaction. For example, a compound may be associated or exhibit one or more characteristics or properties, which may include, by way of example only but is not limited to, one or more characteristics or properties from the group of: an indication of the compound docking with another compound to form a stable complex; an indication associated with a ligand docking with a target protein, wherein the compound is the ligand; an indication of the compound docking or binding with one or more target proteins; an indication of the compound having a particular solubility or range of solubilities; an indication of the compound having particular electrical characteristics; an indication of the compound having a toxicity or range of toxicities; any other indication of a property or characteristic associated with a compound that can be simulated using computer simulation(s) based on physical movements of atoms and molecules; any other indication of a property or characteristic associated with a compound that can be tested by experiment or measured. Further examples of one or more compound property(ies), characteristic(s), or trait(s), may include, by way of example only but are not limited to, one or more of: LogP, Log D, solubility, toxicity, drug-target interaction, drug-drug interaction, off-target drug effects, cell penetration, tissue penetration, metabolism, bioavailability, excretion, absorption, drug-protein binding, drug-lipid interaction, drug-DNA/RNA interaction, metabolite prediction, tissue distribution and/or any other suitable property, characteristic and/or trait in relation to a compound.

Given a property of a compound may include data representative of or indicative of a particular behaviour/characteristic/trait of a compound when a compound undergoes a reaction, this data representative or indicative of the property of the compound may include, by way of example only but is not limited to, any continuous or discrete value/score and/or range of values/score(s), series of values/scores, strings or any other data representative of the property. For example, a property may be associated with, assigned, represented by, or is based on, by way of example only but not limited to, one or more continuous property value(s)/score(s) (e.g. non-binary values), one or more discrete property value(s)/score(s) (e.g. binary values), one or more range(s) of continuous property values/scores, one or more range(s) of discrete property value(s)/score(s), a series of property value(s)/score(s), one or more string(s) of property values, or any other suitable data representation of a property value/score representing a property and the like. The property value/score may be based on measurement data or simulation data associated with the reaction and/or the particular property.

A compound may be assigned a property value/score comprising data representative of whether or not they are associated with a particular property when the compound undergoes a reaction associated with the particular property. This property value/score may be determined or based on, by way of example only but is not limited to, laboratory measurement(s) and/or computer simulated value(s)/score(s). The property value/score assigned to the compound gives an indication of whether that compound is associated with or exhibits the particular property. For example, a compound may be assigned a property value/score depending on whether the compound exhibits a particular property when it undergoes a reaction associated with the particular property. The compound may be said to exhibit the particular property when the property value/score associated with the compound is, by way of example only but is not limited to, above or below a threshold property value/score representing the property, within a region or in the vicinity of a value representative of the property, and the like.

The property model generated for predicting whether a compound has one or more property(ies) according to the invention as described herein may be generated using one or more or a combination of ML techniques. A ML technique may comprise or represent one or more or a combination of computational methods that can be used to generate analytical models and algorithms that lend themselves to solving complex problems such as, by way of example only but is not limited to, prediction and analysis of complex processes and/or compounds. ML techniques can be used to generate ML models (e.g. property models) for use in the drug discovery, identification, and/or optimization in the informatics, cheminformatics and/or bioinformatics fields.

For example, an ML technique may be trained using labelled training datasets to generate a ML model (or property model) for predicting whether a compound has a particular property. A labelled training dataset may include one or more compounds each of which may be labelled with data representative of a known property value/score or label associated with the compound and the particular property. Thus, once the ML technique has trained an ML model based on the labelled training dataset in relation to the particular property, the ML model may predict whether an input compound exhibits a particular property. The ML model may output data representative of a property value/score representing the input compound's association with the particular property. The data representative of the property value/score output by a ML model may be referred to herein as a property prediction value/score. The ML model data representative of one or more compounds may be input to the trained ML model, which may output property prediction values/scores comprising data representative of one or more corresponding property value(s)/score(s) indicative of whether the one or more input compounds are associated or exhibit the particular property.

Examples of ML technique(s) that may be used to generate an ML model or property model for predicting whether a compound has a particular property may include, by way of example only but is not limited to, a least one ML technique or combination of ML technique(s) from the group of: a recurrent neural network; convolutional neural network; reinforcement learning algorithm(s); and any other neural network structure configured for predicting whether a compound has a particular property.

Further examples of ML technique(s) that may be used as described herein according to the invention may include or be based on, by way of example only but is not limited to, any ML technique or algorithm/method that can be trained or adapted to generate one or more candidate compounds based on, by way of example only but is not limited to, an initial compound, a list of desired property(ies) of the candidate compounds, and/or a set of rules for modifying compounds, which may include one or more supervised ML techniques, semi-supervised ML techniques, unsupervised ML techniques, linear and/or non-linear ML techniques, ML techniques associated with classification, ML techniques associated with regression and the like and/or combinations thereof. Some examples of ML techniques may include or be based on, by way of example only but is not limited to, one or more of active learning, multitask learning, transfer learning, neural message parsing, one-shot learning, dimensionality reduction, decision tree learning, association rule learning, similarity learning, data mining algorithms/methods, artificial neural networks (NNs), deep NNs, deep learning, deep learning ANNs, inductive logic programming, support vector machines (SVMs), sparse dictionary learning, clustering, Bayesian networks, representation learning, similarity and metric learning, sparse dictionary learning, genetic algorithms, rule-based machine learning, learning classifier systems, and/or one or more combinations thereof and the like.

Some examples of supervised ML techniques may include or be based on, by way of example only but is not limited to, ANNs, DNNs, association rule learning algorithms, a priori algorithm, case-based reasoning, Gaussian process regression, group method of data handling (GMDH), inductive logic programming, instance-based learning, lazy learning, learning automata, learning vector quantization, logistic model tree, minimum message length (decision trees, decision graphs, etc.), XGBOOST, Gradient Booted Machines, nearest neighbour algorithm, analogical modelling, probably approximately correct learning (PAC) learning, ripple down rules, a knowledge acquisition methodology, symbolic machine learning algorithms, support vector machines, random forests, ensembles of classifiers, bootstrap aggregating (BAGGING), boosting (meta-algorithm), ordinal classification, information fuzzy networks (IFN), conditional random field, anova, quadratic classifiers, k-nearest neighbour, boosting, sprint, Bayesian networks, Naïve Bayes, hidden Markov models (HMMs), hierarchical hidden Markov model (HHMM), and any other ML technique or ML task capable of inferring a function or generating a model from labelled and/or unlabelled training data and the like.

Some examples of unsupervised ML techniques may include or be based on, by way of example only but is not limited to, expectation-maximization (EM) algorithm, vector quantization, generative topographic map, information bottleneck (IB) method and any other ML technique or ML task capable of inferring a function to describe hidden structure and/or generate a model from unlabelled data and/or by ignoring labels in labelled training datasets and the like. Some examples of semi-supervised ML techniques may include or be based on, by way of example only but is not limited to, one or more of active learning, generative models, low-density separation, graph-based methods, co-training, transduction or any other a ML technique, task, or class of unsupervised ML technique capable of making use of unlabeled datasets and/or labelled datasets for training and the like.

Some examples of artificial NN (ANN) ML techniques may include or be based on, by way of example only but is not limited to, one or more of artificial NNs, feedforward NNs, recursive NNs (RNNs), Convolutional NNs (CNNs), autoencoder NNs, extreme learning machines, logic learning machines, self-organizing maps, and other ANN ML technique or connectionist system/computing systems inspired by the biological neural networks that constitute animal brains. Some examples of deep learning ML technique may include or be based on, by way of example only but is not limited to, one or more of deep belief networks, deep Boltzmann machines, DNNs, deep CNNs, deep RNNs, hierarchical temporal memory, deep Boltzmann machine (DBM), stacked Auto-Encoders, and/or any other ML technique.

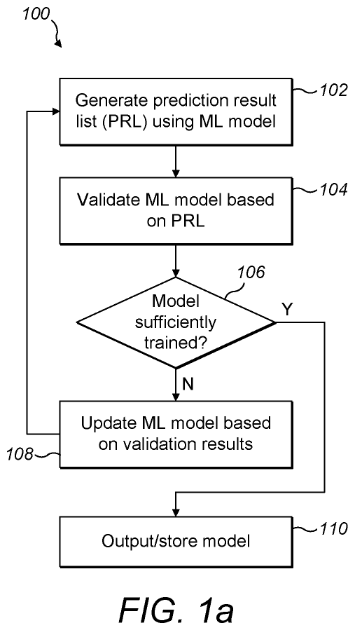

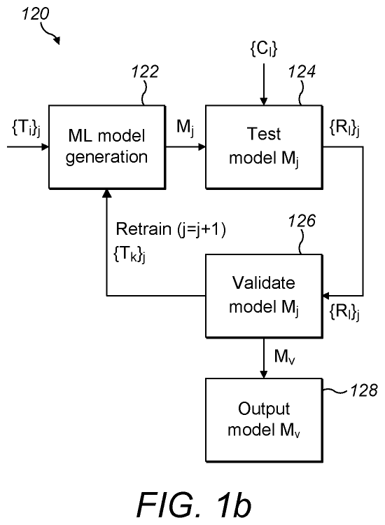

FIG. 1a is a flow diagram illustrating an example process 100 for training a ML technique for generating a ML model for predicting whether a compound exhibits or has a particular property, herein referred to as a property model, according to the invention. The particular property may be based on one of a plurality of properties associated with compounds. The process 100 may use an ML technique that may be trained based on a labelled training dataset, the labelled training dataset including data representative of the relationship or association of a set of compounds with the particular property. The labelled training dataset may have an insufficient number of compound/property associations or may have an insufficient number of dissimilar compound/property associations for training an ML technique to generate a property model that can be used for a broad range of compounds. Thus, the following method further enhances the training of the ML technique for generating an accurate and reliable property model for predicting whether a broad range of compounds have the particular property. The steps of the process 100 may include one or more of the following steps:

In step 102, a prediction result list is generated for a plurality of compounds and their association with the particular property based on the ML model, i.e. the property model. The property model may be generated by training the ML technique based on an initial labelled training dataset, the initial labelled training dataset including data representative of known relationships or associations of a set of compounds with the particular property. A plurality of compounds may include the set of compounds of the labelled training dataset and a further set of compounds in which the association with the particular property is unknown. The plurality of compounds are input to the initially generated property model, which outputs a prediction result list for each of the plurality of compounds that predicts whether that compound has the particular property. The prediction result list may include the plurality of compounds, each of which are mapped to corresponding property prediction values/scores output/estimated by the ML model.

In step 104, the ML model or property model is validated based on the plurality of compounds from the prediction result list having an association with the particular property. The initial labelled training dataset may be used to determine how well the property model predicted the association between each compound of the plurality of compounds and the particular property. This may include determining the model performance statistics or an overall property model score that is indicative of how well the property model predicts the association of the particular property with the compounds. This may further include verifying or further validating the association a selected shortlist of compounds has with the particular property. This can be used to enhance the labelled training dataset.

In step 106, it is determined whether the ML model or property model has been sufficiently trained or whether further training of the property model is necessary. This may be determined based on the property model score (or ML model score) and/or whether there is expected to be a further improvement in the predictive ability of the property model/ML model. If the property model/ML model is determined not to be sufficiently trained (e.g. ‘N’), then the process 100 proceeds to step 108 for updating the property model/ML model, after which steps 102 to 106 may be repeated using the updated property model/ML model until determining the property model/ML model has been validly trained. If the property model/ML model is determined to be sufficiently trained (e.g. ‘Y’) then the process 100 proceeds to step 110.

For simplicity, the term property model is referred to hereinafter and includes, by way of example only but is not limited to, an ML model for predicting whether a compound has or is associated with a particular property (e.g. the particular property may be a property or characteristic associated with compounds and the like). In step 108, the property model may be updated based on the results of the property model validation. For example, an ML score may be used to update the property model. Additionally or alternatively, the property model may be updated based on the results of validating a selected shortlist of compounds. For example, an enhanced or further labelled training dataset may be generated based on the current labelled training dataset, which includes compounds that have a known association with the particular property, and the validation results based on validating whether each of the shortlist of compounds is associated with the particular property. This enhanced or further labelled training dataset may be used to train the ML technique to generate an updated property model that may potentially replace the current property model for predicting whether a compound has the particular property. In any event, once the property model has been updated based on training the ML technique accordingly, the process 100 proceeds to step 102 to determine whether the update property model's performance has improved.

In step 110, once it is determined that the property model has been validly trained, or trained as much as is practicable or possible up to this point, then data representative of the property model may be output for use in predicting whether a compound has a particular property. This may include storing all the parameters, coefficients, weights, hyperparameters and any other data defining the property model and/or how to configure the property model for later use. The output property model may be stored on a computer readable medium, and when it is to be used, it may be retrieved, loaded and executed by one or more processor(s) for predicting whether one or more compound(s) have the particular property.

The ML technique may be initially trained based on a labelled training dataset associated with a subset of the plurality of compounds in relation to the particular property. The labelled training dataset may be further enhanced when validating the property model. This may be achieved by validating a shortlist of compounds from the prediction result list having an association with the particular property. The property model may then be updated based on training the ML technique with a labelled training dataset that includes data representative of the validated shortlist of compounds in relation to the particular property.

In step 108, updating the property model with the additional validated shortlist may include generating a further labelled training dataset that includes data representative of the validated shortlist of compounds associated with the particular property and any previously labelled training dataset associated with the particular property. This may then be used by the ML technique to retrain or update the ML technique based on the further labelled training dataset.

In step 104, validating the shortlist of compounds may include determining, based on certain conditions, whether to perform laboratory experimentation based on the particular property and the shortlist of compounds or whether to perform computer analysis such as, by way of example only but not limited to, simulation analysis based on the particular property and the shortlist of compounds. In response to determining to perform laboratory experimentation, a request may be sent including the shortlist of compounds for laboratory experimentation in relation to the particular property and receive experimental results validating the association of each of the shortlist of compounds with the particular property. The experimental results from the laboratory experimentation may be used to estimate data representative of the association each compound on the shortlist of compounds has with the particular property. This may be used to enhance the labelled training dataset for further updating the property model. In response to determining to perform simulation analysis instead of laboratory experimentation, the shortlist of compounds may be input for computer analysis (e.g. input to a molecular computer simulation in relation to the particular property) for determining the association each shortlist of compounds has with the particular property. The simulation results from the simulation analysis may be used to estimate data representative of the association each compound on the shortlist of compounds has with the particular property. This may also be used to enhance the labelled training dataset for further updating the property model.

Given that laboratory experimentation is typically more costly than computer analysis/simulation, a set of conditions may be required to be met before the shortlist of compounds is sent to a laboratory for determining the association of each compounds with a particular property. The set of conditions may include, by way of example only but are not limited to, one or more from the group of: laboratory experimentation may be selected when a number of validation iterations exceeds a validation iteration threshold in which computer/simulation analysis has been consecutively performed for validating the shortlist; laboratory experimentation may be selected when an indication that laboratory analysis will yield an improvement in an ML score for the property model based on previous property model scores calculated from corresponding prediction result lists generated after each shortlist of compounds has been validated; the number m of selected shortlist of compounds is of a size or number that is cost effective for laboratory experimentation (e.g. the number of m selected shortlist of compounds may be less than 10), where m>=1; or a combination of the number of validation iterations, the indication that laboratory experimentation will provide an improved property model, and the number m or size of the shortlist of compounds.

Computer analysis/simulation may be predominantly selected based on a set of conditions associated with the shortlist of compounds. The computer analysis is used to determine the association of each compound with a particular property. The set of conditions may include, by way of example only but are not limited to, one or more from the group of: computer analysis being selected when a number of validation iterations is less than a validation iteration threshold in which computer/simulation analysis has been consecutively performed for validating the shortlist; computer analysis may be selected when it is determined that computer analysis will still yield an improvement in an ML score for the property model based on previous property model scores calculated from corresponding prediction result lists generated after each shortlist of compounds has been validated; the selected shortlist of compounds is of a size or number m of compounds that is too large to be cost effective for laboratory experimentation (e.g. the number m of selected shortlist of compounds may be in the range of 25 to 500), where m>=1; or a combination of the number of validation iterations, the indication that computer analysis will provide an improved property model, and the size of the selected shortlist of compounds.

Other conditions that may be met for determining whether to perform laboratory experiments may include, by way of example only but is not limited to, determining whether the selected shortlist of compounds has substantially changed from a previously selected shortlist of compounds; in response to determining that the selected shortlist of compounds has not substantially changed from the previously selected shortlist of compounds, electing to perform laboratory experimentation on a selected subset of compounds from the selected shortlist of compounds. The selected subset of compounds may be of a size that is cost effective and/or suitable for laboratory experimentation. The selected shortlist of compounds may be further filtered based on selecting, by way of example only but is not limited to, those compounds in the shortlist that have the most uncertain scores in the prediction result list and/or that are also the most dissimilar compounds compared with compounds in the labelled training dataset.

The property model may be used to predict whether each of a plurality of compounds has a particular property and output these results in the form of a prediction result list. The prediction list may include the one or more compounds mapped to corresponding one or more property prediction values/scores, which may be output by the property model for each compound. Each of the property prediction values/scores given to each compound is indicative of whether that compound is associated with the particular property. This may be achieved by inputting each of the plurality of compounds into the property model and gathering the results output from the property model in a prediction result list. The prediction result list may include, by way of example only but is not limited to, a property prediction score or prediction score for each of the plurality of compounds that indicates whether said each compound has or exhibits the particular property. The plurality of compounds may include a subset of compounds that are in the labelled training dataset use to generate the property model. This allows the quality of the property model to be evaluated and an ML score to be generated. The plurality of compounds also includes a set of compounds that are not in the labelled training dataset used to generate the property model. The prediction result list thus includes prediction scores that predict whether each of a plurality of compounds have or exhibit the particular property.

The prediction result list may be used to select the shortlist of compounds based on the prediction scores (or property prediction values/scores) for each compound and/or the structure of each compound. For example, one or more compounds for the shortlist of compounds may be selected from the prediction result list based on whether a compound has a prediction score indicative of a borderline prediction score. A borderline prediction score is a prediction score that indicates that the property model cannot predict whether a compound has or has not (exhibits or does not exhibit) the particular property. That is, the property model cannot indicate with certainty that the compound is associated with the particular property.

For example, if a compound has or exhibits a particular property then a prediction score or property prediction score/value may have a positive level of certainty represented as a probability in the region of 1 or percentage score in the region of 100% (e.g. in the range of 0.85-1 or in the range of 85-100%). If the compound is known not to have or does not exhibit the particular property then the prediction score for that compound may have a negative level of certainty represented as a probability in the region of 0 or percentage score in the region of 0% (e.g. in the range of 0-0.15 or in the range of 0-15%). Compounds with prediction scores in-between the positive level of certainty and negative level of certainty may be considered to have a prediction score that is uncertain or be borderline. For example, those compounds with prediction scores with probability in the region of 0.5 or having a percentage score in the region of 50% (e.g. between 0.45 and 0.55 or between 45-55%) may be considered to be the most uncertain or the most borderline. That is, the property model cannot determine one way or the other whether these compounds have or have not (exhibit or do not exhibit) the particular property.

Thus, the prediction result list may be filtered to output the compounds that the property model is most uncertain about or cannot predict with certainty their association with the particular property. Thus, a set of compounds based on the most uncertain or borderline cases may be generated from the prediction result list and used in the selection of a shortlist of compounds. For example, the compounds with the most uncertain or borderline prediction scores may be ranked and the M topmost uncertain compounds may be selected for the shortlist. Alternatively or additionally, the set of compounds based on the most uncertain or borderline cases may be further filtered by generating a set of the most uncertain dissimilar compounds. The shortlist of compounds may be selected based on selecting, from a ranked list of uncertain or borderline compounds, a number of m<=M compounds that are the most structurally dissimilar to the compounds that have a prediction score with a positive or negative level of certainty. Alternatively or additionally, the shortlist of compounds may be based on selecting from the ranked list of uncertain or borderline compounds those compounds that are the most structurally dissimilar to the compounds that make up the labelled training dataset used to generate the property model. Selecting the shortlist of compounds based on this method may prevent the retraining or update to property model from overfitting or focussed on a particular type or structure of compound and will allow the training of the ML technique to generate a property model that can make predictions for a broad range of structurally similar and dissimilar compounds.

FIG. 1b is a schematic diagram illustrating an example training apparatus or system 120 for implementing the example process 100 of FIG. 1a according to the invention. The training apparatus/system 120 includes a machine learning (ML) model generation (MLG) device 122, a Model Testing (MT) device 124, and a validation model (VM) device 126 that are coupled together in a feedback loop, which may be iterated or repeated until an property model is considered to be validly trained. The training apparatus 120 may be configured to implement the process 100 of FIG. 1a. Each of the components/devices 122, 124 and 126 of the training apparatus 120 may be configured to iteratively implement one or more steps of the process 100 of FIG. 1a as described above for iteratively training the ML technique to generate an improved, accurate and reliable property model for predicting whether a compound is associated with a particular property.

Initially, for the first iteration (e.g. j=1), the MLG device 122 receives a labelled training dataset {Tᵢ}ⱼ for 1<=i<=N, where N is the number of training data elements (e.g. in the region of 1000s or more) in which the i-th training data element includes data representative of a compound Cᵢ and its known association with the particular property. The MLG device 122 trains a ML technique (this may be predetermined) using the labelled training dataset {Tᵢ}ⱼ to generate a property model Mⱼ for the j-th iteration. The property model Mⱼ predicts whether an input compound Cₗ has a particular property. The labelled training dataset {Tᵢ}ⱼ may incorporate further training data {Tₖ}ⱼ based whether the VM device 126 considers further training is necessary and outputs validation results or further training data {Tₖ}ⱼ that may be used to enhance labelled training dataset {Tᵢ}ⱼ for training the ML technique to generate an updated property model Mⱼ in the next iteration (e.g. j=j+1).

In the j-th iteration, the MT device 124 receives the generated property model Mⱼ, inputs a plurality of compounds {Cₗ}ⱼ to the property model Mⱼ, where 1<=k=L and L is the number of the plurality of compounds, and output a prediction result list {Rₗ}ⱼ for 1<=k=L, where the l-th prediction result R_{l,j} for the j-th iteration may include, by way of example only but is not limited to, data representative of the compound Cₗ and a prediction score P_{l,j} for the j-th iteration. The prediction score P_{l,j} being a value that represents the property model's Mⱼ prediction that compound Cₗ is associated with the particular property. The prediction result list {Rₗ}ⱼ predicts whether each of the plurality of compounds {Cₗ}ⱼ has the particular property. For each iteration j, the number of the plurality of compounds {Cₗ}ⱼ may or may not change depending on whether it is required for the property model Mⱼ to be further trained over a broader range of compounds or not.

The VM device 126 receives, at least, the prediction result list {Rₗ}ⱼ and uses this to validate whether the property model Mⱼ is validly trained or requires further training. The VM device 126 may also receive a property model score Sⱼ for the j-th iteration for the j-th feedback loop. Alternatively or additionally, the VM device 126 may generate a property model score Sⱼ for the j-th iteration of the feedback loop based on the prediction result list {Rₗ}ⱼ and/or labelled training dataset {Tᵢ}ⱼ. The property model score Sⱼ may be stored and monitored for each iteration of the feedback loop. The property model score Sⱼ and/or the prediction result list {Rₗ}ⱼ may be used to determine, by way of example only but is not limited to, a) whether further training of the property model Mⱼ is required as described with reference to process 100 and FIG. 1a; b) whether to validate a shortlist of compounds using computer analysis/simulation or using laboratory experimentation as described with reference to process 100 and FIG. 1a; c) whether to increase or decrease the number of compounds in the shortlist of compounds as described with reference to process 100 and FIG. 1a; d) whether to change the selection of compounds from the prediction result list {Rₗ}ⱼ as described with reference to process 100 and FIG. 1a.

The VM device 126 may determine, based on the ML score Sⱼ and/or previous ML score(s) {Sₖ} for 1<=k<j, that property model Mⱼ should be updated and further training of ML technique is necessary (e.g. step 106 of process 100). This may include selecting a shortlist of compounds that may be validated using either computer analysis or laboratory experimentation. The VM device 126, as a result, may output further training data {Tₖ}ⱼ and/or validation results that may be used to generate further training data {Tₖ}ⱼ in relation to the selected shortlist of compounds. The MLG device 122 may use the further training data {Tₖ}ⱼ or incorporate the further training data {Tₖ}ⱼ into the labelled training dataset {Tᵢ}ⱼ for the next iteration of the feedback loop (e.g. j=j+1). Thus, the further training data {Tₖ}ⱼ may be used to enhance the labelled training dataset {Tᵢ}ⱼ for training the ML technique to generate an updated property model Mⱼ on the next iteration when j=j+1 and the process 100 and its steps implemented by components/devices 122, 124 and 126 are repeated.

This iterative process 100 may continue until the VM device 126 considers the updated property model Mⱼ has been sufficiently trained. Once the property model Mⱼ has been sufficiently trained, the property model Mⱼ is considered to be a validly trained property model Mᵥ for predicting whether a compound is associated with a particular property. The output device 128 may generate data representative of the valid property model Mᵥ for storing the property model Mᵥ and/or for using property model Mᵥ to predict whether a compound is associated with a particular property.

As can be seen, the process 100 can be used to train a ML technique to generate an property model based on labelled training dataset. This may also be termed training or updating the property model. The property model is the model artifact of data embodying the property model that is created by the training process 100 resulting in an property model Mᵥ that is configured for predicting whether a compound (e.g. a new compounds) is associated with the particular property. The prediction score for the compound may indicate whether the compound has the particular property or not, or how uncertain the property model's prediction is in relation to whether the compound is associated with the particular property.

The output device 128 may output data representative of property model Mᵥ may include, by way of example only but is not limited to, the hyperparameters used to train the ML technique, the weights, coefficients, parameters that are generated during training the ML technique, any other data that defines the structure of property model Mᵥ or that is required for implementing property model Mᵥ on one or more apparatus, computing systems, devices and/or processor(s) and the like to enable property model Mᵥ to predict whether a compound is associated with a particular property. The property model Mᵥ may be stored for retrieval and used to predict whether a compound is associated with a particular property.

The training apparatus or system 120 for generating the property model for predicting whether a compound is associated with a particular property, may be based on a functional or modular components/modules that may be implemented in software and/or hardware. The system 120 may include a model generation module for training a ML technique to generate the property model; a model test module for generating a prediction result for a compound and their association with the particular property using the property model; a validation module for validating the property model based on the compound from the prediction result having an association with the particular property; and a model update module for updating the property model based on the property model validation. These modules may be further modified and/or configured to implement method/process 100 and/or the method(s)/process(es) as described herein.

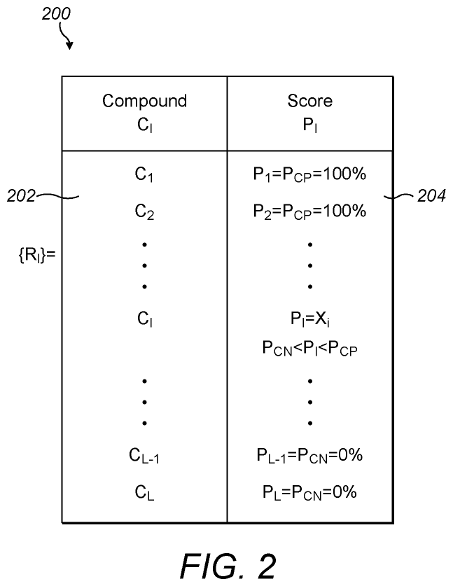

FIG. 2 is a table illustrating an example prediction result list {Rₗ}ⱼ 200 for 1<=k=L output from a property model for predicting whether a plurality of compounds {Cₗ} for 1<=k=L are associated with a particular property according to the invention. The property prediction value/score indicating a compound's association with a particular property Cₗ may include data representative of a prediction scores Pₗ. The prediction result list {Rₗ}ⱼ 200 includes data representative of the plurality of compounds {Cₗ} 202 and their corresponding prediction scores {Pₗ} 204 (e.g. property prediction values/scores) for 1<=l<=L. The plurality of compounds {Cₗ} includes compounds C₁, C₂, . . . , Cₗ, . . . , C_{L-1}, C_{L}. The corresponding plurality of prediction scores {Pₗ} 204 includes prediction scores P₁, P₂, . . . , Pₗ, . . . , P_{L-1}, P_{L}. Each prediction score Pₗ indicates whether said each compound Cₗ has or is associated with the particular property. The validation step 106 may select a shortlist of compounds from the prediction result list {Rₗ}ⱼ 200 based, at least in part, on the prediction scores.

As described previously, the prediction score comprises or represents data representative of a value representative or indicative of the ML Model predicting whether a compound has or has not a particular property. The prediction score may be a value, by way of example only but not limited to, a probability value, a certainty value or score, a percentage score or any other value that is indicative of representing the prediction of whether a compound has or has not the particular property, or a prediction of whether the compound exhibits or does not exhibit the particular property, and/or a prediction of how associated the compound is with the particular property; and/or any other value, score or statistic that is useful for assessing or classifying whether a compound is associated with a particular property and the like.

For example, the prediction score Pₗ for whether compound Cₗ is associated with a particular property may be represented as a certainty score value. Compounds that are known to have the particular property are given a value representing “positive” certainty score (e.g. P_{CP}). Compounds that are known not to have the particular property are given a value representing a “negative” certainty score (e.g. P_{CN}). Other compounds are given a value representing an “uncertainty” score (Pₗ=Xₗ, where P_{CN}<Xₗ<P_{CP}). The “uncertainty” score may be a continuous real value that represents the level of uncertainty the ML Model has in relation to whether that compound is associated with the particular property. The “uncertainty” score may have a continuous value that is between the value representing the positive certainty score and the value representing the negative certainty score (e.g. P_{CN}<Pₗ<P_{CP}). In the present example, the certainty score is represented as a percentage certainty score, where the positive certainty score is 100%, the negative certainty score is 0%, and the uncertainty score is between the positive and negative certainty scores i.e. between 0% and 100%.

In FIG. 2, the prediction result list {Rₗ}ⱼ 200 ranks the plurality of compounds {Cₗ} 202 based on their prediction scores {Pₗ} 204. For example, if a compound has or exhibits a particular property then the prediction score may have a positive level of certainty represented as a probability in the region of 1 or percentage score in the region of 100% (e.g. in the range of 0.85-1 or in the range of 85-100%). In FIGS. 2, C₁ and C₂ have positive certainty scores represented as a percentage score of P_{CP}=100%, which means that the ML Model is 100% confident that these compounds C₁ and C₂ have the particular property. As well, C_{L-1} and C_{L} have negative certainty scores represented as a percentage score of P_{CN}=0%, which means that the ML Model is 100% confident that these compounds C_{L-1} and C_{L} do not have the particular property. There may be one or more or a plurality of compounds {Cₗ} in which the prediction score has a value Pₗ=Xₗ that is between P_{CN}<Pₗ<P_{CP}, where the ML Model has a continuum of confidence as to whether these compounds are associated with particular property. Of interest are those compounds located in a region midway between P_{CN} and P_{CP} (e.g. 45%<Pₗ<55%), which include compounds that the property model predicts as being most uncertain as to whether these compounds are or are not associated with the particular property. It is these compounds that may be of interest for selecting in a shortlist of compounds that may be validated in relation to the particular property.

As an example, if the compound is reasonably known to have or does exhibit the particular property, then the prediction score Pₗ for that compound may have a positive level of certainty represented as a probability in the region of 1 or a percentage score in the region of 100% (e.g. a probability in the range of 0.85-1 or a percentage score in the range of 85-100%). If the compound is reasonably known not to have or does not exhibit the particular property, then the prediction score Pₗ for that compound may have a negative level of certainty represented as a probability in the region of 0 or percentage score in the region of 0% (e.g. a probability in the range of 0-0.15 or a percentage score in the range of 0-15%). Compounds with prediction scores in between the positive level of certainty and negative level of certainty may be considered to have a prediction score that is uncertain or be borderline. For example, those compounds with prediction scores with probability in the region of 0.5 or having a percentage score in the region of 50% (e.g. between 0.45 and 0.55 or between 45-55%) may be considered to be the most uncertain or the most borderline. That is, the property model cannot determine one way or the other whether these compounds have or have not (exhibit or do not exhibit) the particular property. It is these compounds that will be of interest to validate in relation to the particular property and so generate further labelled training datasets for updating the property model as described herein.

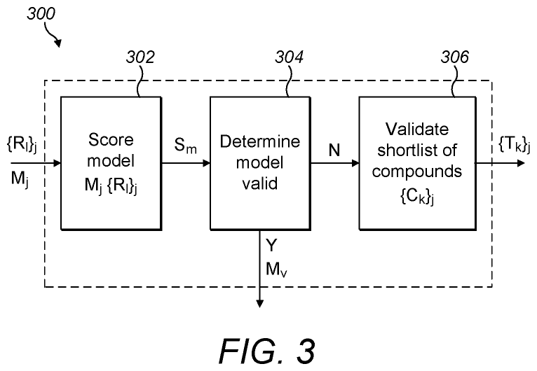

FIG. 3 is a schematic diagram illustrating an example validation apparatus 300 for validating an property model in each iteration j of process 100 according to the invention. The validation apparatus 300 receives a prediction result list {Rₗ}ⱼ 200, which may be used by a score generator 302, model validator 304, and shortlist validator 306. The score generator 302 calculates a property model score Sⱼ based on the received prediction result list {Rₗ}ⱼ 200. The model validator 304 may use the property model score Sⱼ to determine whether the property model is validly trained based on property model score Sⱼ and any previously generated property model scores {Sₖ} for 1<=k<j. The property model score Sⱼ is an indication of how well the property model predicts whether compounds are associated with the particular property. If the Model Validator 304 considers further training is required, i.e. property model is not validly trained (e.g. ‘N’), then shortlist validator 306 selects a shortlist of compounds that should enhance the property model (e.g. as described herein in relation to FIGS. 1a-2) and then validates the shortlist of compounds in relation to the particular property. The shortlist validator 306 outputs validation results, which in this example are in the form of further training data elements {Tₖ}ⱼ, which can be used by the ML technique in generating/updating the property model in the next iteration j=j+1 of process 100.

The score generator 302 may use labelled training dataset {Tᵢ}ⱼ and received prediction result list {Rₗ}ⱼ 200 for calculating a property model score Sⱼ indicative of the performance of the property model for the j-th iteration. The property model score Sⱼ may be calculated based on model performance statistics that can be estimated from labelled training dataset {Tᵢ}ⱼ and/or received prediction result list {Rₗ}ⱼ 200. Model performance statistics may comprise or represent an indication of the performance of a property model based on labelled training dataset {Tᵢ}ⱼ and/or received prediction result list(s){Rₗ}ⱼ 200. The model performance statistics for a property model may be based on, by way of example, but is not limited to, one or more from the group of: positive predictive value or precision of the property model; sensitivity, true predictive rate, or recall of the property model; a receiver operating characteristic, ROC, graph associated with the property model; an area under a precision and/or recall ROC curve associated with the property model; any other function associated with precision and/or recall of the property model; and any other model performance statistic(s) for use in generating a property model score Sⱼ indicative of the performance of the property model.

The model validator 304 may use the property model score Sⱼ to determine whether the property model has been validly trained or whether property model requires further training. The model validator 304 may use previous or historical property model score(s) {Sₖ} for 1<=k<j to determine whether further improvements in the quality of property model may be possible. The model validator 304 may also, by way of example only but is not limited to, keep track of the number of iterations j that have been completed; keep track of the number of consecutive times a shortlist has been validated using computer analysis; keep track of the number of times a shortlist has been validated using laboratory experiments; keep track of the number of uncertain compounds in the received prediction result list(s){Rₗ}ⱼ 200. These measures are useful to determine whether further improvements in the quality of property model may be possible.

For example, if the property model score(s) Sⱼ and {Sₖ} for 1<=k<j have plateaued; the number of consecutive times a selected shortlist has been validated using computer analysis/simulations is greater than a predetermined threshold; and there has not been any validation of a selected shortlist of compounds using laboratory experiments; then the model validator 304 may determine that further improvements are possible if a selected shortlist of compounds are validated using laboratory experimentation. Thus, it may indicate to the shortlist validator 306 that further training is necessary and that the shortlist is selected for use in being validated using laboratory experimentation rather than computer analysis/simulation.

In another example, if the property model score(s) Sⱼ and {Sₖ} for 1<=k<j have not plateaued but seem to be increasing; the number of consecutive times a selected shortlist has been validated using computer analysis/simulations is less than a predetermined threshold; and there has not been any validation of a selected shortlist of compounds using laboratory experiments; then the model validator 304 may determine that further improvements are still possible using a selected shortlist of compounds being validated using computer analysis/simulation. Thus, it may indicate to the shortlist validator 306 that further training is necessary and that the shortlist is selected for use in being validated using computer analysis/simulation.

In a further example, if the property model score(s) Sⱼ and {Sₖ} for 1<=k<j have decreased; the number of consecutive times a selected shortlist has been validated using computer analysis/simulations is less than a predetermined threshold; and there has not been any validation of a selected shortlist of compounds using laboratory experiments; then the model validator 304 may determine that further improvements are possible if a selected shortlist of compounds are validated using laboratory experimentation. Thus, it may indicate to the shortlist validator 306 that further training is necessary and that the shortlist is selected for use in being validated using laboratory experimentation rather than computer analysis/simulation.

The shortlist validator 306 may receive an indication from the model validator 302 that further training is required. The shortlist validator 306 may also, by way of example only but is not limited to, keep track of the number of iterations j that have been completed; keep track of the number of consecutive times a shortlist has been validated using computer analysis; keep track of the number of times a shortlist has been validated using laboratory experiments; keep track of the number of uncertain compounds in the received prediction result list(s){Rₗ}ⱼ 200. These measures may be sent to the model validator 302 for assisting it in making its decisions in relation to the validity of the property model at iteration j. They may also be useful to determine the type and/or number of shortlist of compounds that may be selected to maximise the chances that the quality of an updated property model based on the validation results may be enhanced or improved. Alternatively or additionally, the shortlist validator 306 may receive an indication that validation of the shortlist should be performed based on computer analysis/simulation or via laboratory experimentation.

The shortlist validator 306 may select an appropriate shortlist of compounds as described herein or in relation to FIGS. 1a to 2 and 4a-5 and have the selected shortlist of compounds validated in relation to the particular property via the selected validation method of either computer analysis or laboratory experimentation. The shortlist validator 306, as a result, may output the validation results as further training data {Tₖ}ⱼ. As described, the further training data {Tₖ}ⱼ may be used or incorporated into the labelled training dataset {Tᵢ}ⱼ for updating the property model by the ML technique in the next iteration of the feedback loop (e.g. j=j+1).

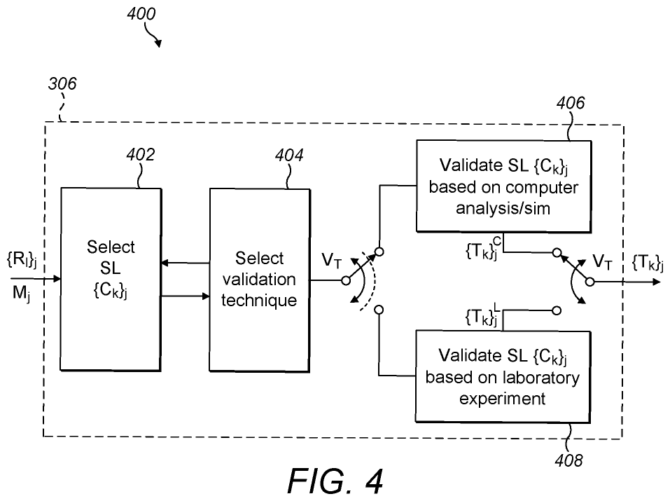

FIG. 4 is a schematic diagram illustrating an example validation apparatus 400, which may be used in place of shortlist validator 306, for selecting and validating a shortlist of compounds for use in training a ML technique to generate or update the property model according to the invention. The validation apparatus 400 includes a shortlist selector 402, a validation selector 404, computer analysis validator 406 and laboratory validator 408. Validation apparatus 400 receives at least a prediction result list {Rₗ}ⱼ 200 and the shortlist selector 402 selects from the prediction result list prediction result list {Rₗ}ⱼ 200 a shortlist of compounds {Cₖ}ⱼ, which when validated in relation to the particular property, should enhance the update of the property model Mⱼ on the next iteration of the training process 100.

As described with reference to FIG. 2, the shortlist of compounds {Cₖ}ⱼ that are of interest may include those that require further validation in relation to the particular property and can be used to enhance the accuracy and reliability of the property model if selected correctly or judiciously. The shortlist of compounds may be selected from the prediction result list {Rₗ}ⱼ 200 based, at least in part, on the prediction scores {Pₗ}. The compounds of interest in the prediction result list {Rₗ}ⱼ 200 are those that are considered to be the most uncertain or the most borderline based on their prediction scores. For these compounds, the property model may not be able to determine one way or the other whether these compounds have or have not (exhibit or do not exhibit) the particular property (e.g. the prediction score is generally between 0.45 and 0.55 or between 45-55%). However, any other prediction score Pₗ satisfying P_{CN}<Pₗ<P_{CP} may also be useful as being selected as part of the shortlist of compounds.

The shortlist selector 402 may select compounds from a ranked prediction result list {Rₗ}ⱼ 200 that has been ranked such that the topmost compounds in the list are ones in which the property model is most uncertain of. Generating a ranked list of compounds that the property model is unable to predict as having or not having the particular property will assist in selecting a shortlist of compounds {Cₖ}ⱼ that will enhance the training of the ML technique to generate more accurate and reliable property models. The ranked list may be generated in the following manner.

Assume that the maximum prediction score the property model Mⱼ may give for all compounds it predicts as having the particular property is X (e.g. a positive certainty score, probability 1, or percentage score of 100%) and the minimum prediction score for all compounds it predicts as definitely not having the particular property is Y (e.g. a negative certainty score, probability of 0, or percentage score of 0%), where X>Y. For each compound C, input to the property model Mⱼ, also assume that the property model outputs a prediction score Pₗ in the range of Y<=Pₗ<=X, which provides an indication of how certain the property model is in its prediction that compound has or has not the particular property. The prediction result list {Rₗ}ⱼ 200 may be used to generate a ranked list of compounds that the property model is most uncertain of, ranking from the most uncertain prediction score to the most certain prediction score with positive or negative level of certainty. Let Pₗ be the prediction score for the l-th compound in the prediction result list {Rₗ}ⱼ 200, for 1<=l<=L. The compounds with prediction scores P_{I}>(X+Y)/2 may be given a ranked score S_{Rl} by subtracting their prediction score Pₗ from X, i.e. S_{Rl}=X−Pₗ. The compounds with prediction scores Pₗ<=(X+Y)/2 may be given a ranked score S_{Rl}=Pₗ. Thus, the l-th compound Cₗ of the prediction result list has a ranked score Rₗ=X−Pₗ when Pₗ>(X+Y)/2 or a ranked score Rₗ=Pₗ when Pi<=(X+Y)/2. Thus, ranking the prediction result list {Rₗ}ⱼ 200 in descending order of the ranked score S_{Rl} will produce a ranked list of compounds with the topmost compounds being compounds that the property model is most uncertain about.

The shortlist selector 402 may select one or more compounds for the shortlist of compounds from the prediction result list {Rₗ}ⱼ 200 based on whether a compound has a prediction score indicative of a borderline prediction score. In the above case, generating a ranked list of compounds from the prediction result list {Rₗ}ⱼ 200 that ranks the topmost compounds being compounds that the property model is most uncertain about will assist in identifying the most uncertain compounds that should be in the shortlist of compounds. These topmost compounds may be used to select one or more compounds for the shortlist of compounds, which means selecting one or more compounds from the prediction result list {Rₗ}ⱼ 200 having an uncertain prediction result.

Although the topmost compounds in the ranked list of compounds may assist in enhancing the training of the ML technique and generation/update of the property model, some of these may be too structurally similar to the compounds that have already been used for training the ML technique and generating/updating the property model Mj. In addition or alternatively to selecting the topmost uncertain compounds from the ranked list of compounds, the shortlist may be generated by selecting one or more compounds that are structurally dissimilar to the compounds used in any labelled training data used so far; or selecting one or more compounds that are structurally dissimilar from each other in the topmost compounds of the ranked list of uncertain compounds. Furthermore, the shortlist may be generated by selecting one or more of the topmost compounds from the ranked list that are structurally dissimilar to the compounds used in any labelled training data used so far.

The validation selector 404 may be configured to select a validation technique for validating the selected shortlist of compounds in relation to the particular property. As described with reference to FIG. 3, the validation selector may also, by way of example only but is not limited to, keep track of the number of compounds selected in the shortlist of compounds {Cₖ}ⱼ; keep track of the type or number of dissimilar compounds in the shortlist of compounds; keep track of the number of iterations j that have been completed; keep track of the number of consecutive times a shortlist has been validated using computer analysis/simulation; keep track of the number of times a shortlist has been validated using laboratory experiments; keep track of the number of uncertain compounds in the received prediction result list(s) {Rₗ}ⱼ 200; and keep track of the property model score Sⱼ. These measures may be used to determine whether to select computer analysis/simulation for validating the shortlist or whether to select laboratory experimentation for validating the shortlist. They may also be useful to determine the type and/or number of shortlist of compounds {Cₖ}ⱼ that may be selected to maximise the chances that the quality of an updated property model based on the validation results may be enhanced or improved.

For example, the validation selector 404 may determine to perform computer analysis/simulation based on one or more from the group of: a number of validation iterations exceeding a validation iteration threshold in which simulation analysis has been consecutively performed for validating the shortlist, where the number of validation iterations in which simulation analysis is performed consecutively is greater than the number of validation iterations in which laboratory analysis is performed; an indication that simulation analysis will yield an improvement in an ML score for the property model based on previous property model scores calculated from corresponding prediction result lists generated after each shortlist of compounds has been validated; or a combination on a number of validation iterations and an indication that computer analysis/simulation will provide an improved property model.

Furthermore, the number of compounds that can be validated in relation to a particular property using computer analysis/simulation largely depends on the computational resources available. Typically, the number of compounds that may be simulated in a reasonable amount of time may be between 50-500 compounds (e.g. 50-100). It is to be appreciated that the number of compounds that can be simulated in relation to a particular property is dependent on the computational resources available, and that the number of compounds that can be simulated will increase as computational resources increase and become cheaper and faster. Typically, the number of compounds m that may be validated in relation to the particular property using laboratory experimentation is in the order of 4 to 10 compounds, e.g. 6-8 experiments. This is because it is costly in terms of laboratory hours to run the experiments and costly in terms of the expense required. Thus, if validation is being performed using computer analysis/simulation, then the number of compounds m in the shortlist of compounds may be selected to be one, two or several orders of magnitude larger than the number of compounds m in the shortlist of compounds that may be used when being validated using laboratory experiments. Thus, the validation selector 404 and the shortlist selector 402 may communicate with each other, to determine the maximum size of the shortlist of compounds {Cₖ}ⱼ that may be validated. Alternatively, the shortlist selector 402 may simply send the shortlist of compounds to the validation selector 404 and based on which validation method is selected, the validation selector 404 may truncate, if necessary, the shortlist of compounds {Cₖ}ⱼ to ensure an appropriate number of compounds is validated by the selected validation method (e.g. computer analysis/simulation or laboratory experimentation).

For example, the validation selector 404 may be configured to indicate, via a selector V_{T} or some other technique/method, that computer analysis/simulation be selected such that the shortlist of compounds {Cₖ}ⱼ is directed/requested to be processed by the computer analysis validator 406, which is used to validate the shortlist of compounds. The computer analysis validator 406 may be connected to one or more computer analysis/simulation systems (e.g. Molecular Dynamics (MD) (RTM) molecular simulator) that can atomistically simulate whether a compound has or exhibits a particular property. For example, MD simulator simulates the properties of compounds/molecules using atomistic and/or physical simulation of the molecules. The types of properties of compounds that may be simulated by MD includes, by way of example only but is not limited to, docking simulations including protein docking with the compound, and/or any other property or compound that can be simulated to determine whether the compound has the particular property.

The computer analysis/simulator validator 406 validates the shortlist by sending the shortlist to a computer analysis/simulation system that performs a computer analysis/simulation analysis based on the particular property and the shortlist of compounds {Cₖ}ⱼ. The computer analysis/simulator validator 406 may receive the computer analysis/simulation results from the computer analysis/simulation system. The computer analysis/simulation results may be used to estimate the association each compound on the shortlist of compounds has with the particular property. The computer analysis/simulation results associated with the short list of compounds {Cₖ}ⱼ may be output in the form of a labelled training dataset {Tₖ}ⱼ^{C}, which may be used to generate a further training dataset {Tₖ}ⱼ for use, as described herein, by ML technique in generating/updating the property model Mⱼ for the next iteration of the process 100. The selector V_{T} may be used to select the labelled training dataset {Tₖ}ⱼ^{C} as the further training dataset {Tₖ}_{J} for training the ML technique to generating/updating the property model Mⱼ for the next iteration of process 100.

In another example, the validation selector 404 may be configured to indicate, via a selector V_{T} or some other technique/method, that laboratory experimentation be selected such that the shortlist of compounds {Cₖ}ⱼ is directed/requested to be processed by the laboratory validator 408 for validating the shortlist of compounds. The laboratory validator 408 may be connected to one or more computer systems associated with one or more laboratory(ies) that can receive the shortlist of compounds and perform laboratory experiments in relation to whether each compound in the shortlist has or exhibits the particular property. The experimental results associated with the short list of compounds {Cₖ}ⱼ may be output in the form of a labelled training dataset {Tₖ}ⱼ^{L}

Alternatively, the laboratory validator 408 may notify an operator with the shortlist of compounds and the particular property for laboratory experiments. The operator may send the shortlist of compounds and request a laboratory to perform experiments to determine whether each of the shortlist of compounds has or exhibits the particular property. After the experiments have concluded, the experimental results and/or further training data associated with the shortlist of compounds and whether each have or are associated with the particular property may be sent to the laboratory validator 408.

The laboratory validator 408 may, on receiving experimental results or training data in relation to the shortlist of compounds and their association with the particular property, be configured to output a labelled training dataset {Tₖ}ⱼ^{L} based on the experimental results corresponding to the shortlist of compounds. The labelled training dataset {Tₖ}ⱼ^{L} may be used as further training data {Tₖ}ⱼ for use, as described herein, by ML technique in generating/updating the property model Mⱼ for the next iteration (e.g. j=j+1) of the process 100. The selector V_{T} may be used to select the labelled training dataset {Tₖ}ⱼ^{L} as the further training dataset {Tₖ}ⱼ for training the ML technique to generating/updating the property model Mⱼ for the next iteration of process 100.

Although the selector V_{T} is shown as a switching circuit, switching between computer analysis/simulator validator 406 and laboratory validator 408, this is by way of example only and the invention is not so limited, it is to be appreciated that the skilled person may use any other method, technique, apparatus, or hardware/software for selecting between and/or directing/requesting the shortlist of compounds to be processed in relation to the particular property by computer analysis/simulator validator 406 and/or laboratory validator 408.

Further considerations by the validation selector 404 for determining whether to perform laboratory experimentation may be based on one or more from the group of: a number of validation iterations exceeding a validation iteration threshold in which simulation analysis has been consecutively performed for validating the shortlist; an indication that laboratory analysis will yield an improvement in an ML score for the property model based on previous property model scores calculated from corresponding prediction result lists generated after each shortlist of compounds has been validated; and or a combination on a number of validation iterations and an indication that laboratory experimentation will provide an improved property model.

Although a set of selection and/or validation rules may be derived for selecting a shortlist of compounds and/or selecting a validation method as described herein for validating the shortlist of compounds, a selection model may instead be generated based on training a reinforcement learning technique. The selection model is for predicting a shortlist of compounds suitable for validation in relation to the particular property. Thus, instead of using a set of selection rules to select an appropriate shortlist of compounds that the property model is uncertain about, an RL technique may be trained over time to make this selection. Once the RL technique has learnt to select a shortlist of compounds for enhancing the property model, the generated selection model may be used for training property models that are used to predict whether a compound exhibits or has a different property to the particular property. This is because the selection model does not depend on the type of property that each property model is modelling to predict.

An RL technique can be trained to learn what compounds from a result prediction list to select in order to maximise the quality of selection and generate a selection model. The quality of selection is maximised when the selected shortlist of compounds are the best compounds to pick from that particular result prediction list, that when validated in relation to the particular property to maximise quality of the resulting updated property model. RL technique may be used to iteratively train a selection model that is robust enough to select the most appropriate or best shortlist of compounds from a result prediction list for validation in relation to the particular property. The training process for the selection model may be based on the following:

Initially, in the first iteration (e.g. j=1) of the ML training process, the property model may be generated by training a ML technique based on a first set of labelled training dataset. The first set of the labelled training dataset may be used to train the ML technique to generate the property model whilst a second set of the labelled training dataset may be held aside for evaluating the quality of the property model. Once the property model has been trained by the ML technique, the second set of the labelled training dataset is input to the property model and a prediction result list is output. As well, a property model score Sⱼ may be derived for evaluating the quality of the property model based on the prediction result list and/or the second set of labelled training dataset. The RL technique can be taught which compounds of the prediction result list may be the best to select for validation and thus generates a selection model. Initially, the selection model being trained by the RL technique may select a “random” set of compounds from the result prediction list as the shortlist of compounds. The selection model training process proceeds to the next iteration (e.g. j=j+1).

In the second iteration (e.g. j=2), the property model may be retrained based on the first set of labelled training dataset and the selected portion of the second set of the labelled training dataset corresponding to the selected shortlist of compounds selected by the selection model being trained by the RL technique in the previous iteration. Once the property model has been retrained or updated by the ML technique, the second set of the labelled training dataset is input to the property model and a prediction result list is output. Another property model score Sⱼ may be derived for evaluating the quality of the property model based on the prediction result list and/or the second set of labelled training dataset. The property model score {Sₖ} 1<=k<j from a previous iteration (e.g. k=j−1) may be compared with the property model score Sⱼ of the current iteration. The retrained or updated property model may then be retained/kept for another iteration of training the selection model. If there is an improvement in quality/accuracy in the performance of the property model then this is fed back to the RL technique as a reward. The selection model associated with the RL technique may be updated/retrained based on the reward. The selection model is then used to select another set of compounds from the result prediction list as the shortlist of compounds for validation. The selection model training process proceeds to the next iteration (e.g. j=j+1).

However, if the comparison results in there not being an improvement in quality/accuracy in the performance of the property model then this is fed back to the RL technique as a penalty. The selection model associated with the RL technique may be updated/retrained based on the penalty. Given that the property model has worsened in performance, it may be reverted back to a previous retained/kept property model to before the property model had poor performance. The selection model may then be used to select another set of compounds from the result prediction list as the shortlist of compounds for validation. The selection model training process proceeds to the next iteration (e.g. j=j+1).

Once the ML scores {Sₖ} 1<=k<=j indicate that the performance of the ML technique has plateaued, then it may be assumed that the selection model has been trained. The property model may then be further trained as described with reference to FIGS. 1a-4 in which a plurality of compounds, most of which the property model has not seen before, may be input to the property model to generate a prediction result list in which the selection model may be used to select a shortlist of compounds for validation. As described, the validation results may be used to further update the property model and thus iteratively further improve the property model. In this process (e.g. process 100), the selection model may also be further trained based on the above-mentioned training selection process but in which each selected shortlist of compounds is validated using computer analysis/simulation, and/or on the rare occasion using laboratory experimentation. ML scores may be calculated to allow the RL technique to reward or penalise the selection model during retraining.

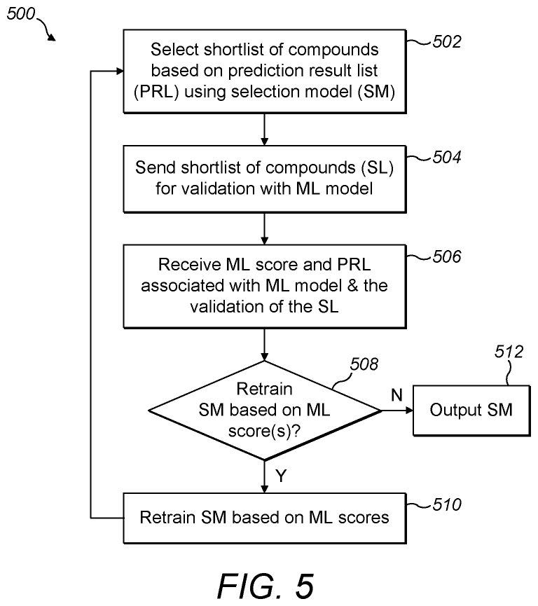

FIG. 5 is a flow diagram illustrating another example process 500 for training a selection model to selecting a shortlist of compounds for use in FIGS. 1a-4 according to the invention. The selection model may initially be trained by a RL technique as described previously in which a first portion of the labelled training dataset is used to train the property model and a second portion of the labelled training dataset is used to evaluate the property model to generate a prediction result list and an property model score Sⱼ for initially training the RL technique to generate/retrain a selection model.

The process 500 may include the following steps for training or retraining an RL technique to generate a selection model that may better predict a shortlist of compounds based on a result prediction list output from a property model Mj and/or a property model score Sj. In step 502, the selection model may be used to select a set of compounds for the shortlist of compounds from a prediction result list output from the property model Mj for validation of the shortlist of compounds. In step 504, the selection model sends the selected shortlist of compounds for validation.

Computer analysis/simulation may be used to validate whether each of the selected shortlist of compounds has the particular property. On occasion, it may be determined, as described herein, to validate some or all of the selected shortlist of compounds via laboratory experimentation. The property model may be updated based on the ML technique, the labelled training dataset and also the validated shortlist of compounds. That is, the validated shortlist of compounds may be represented as further labelled training dataset associated with the shortlist of compounds, which may be used to further train the ML technique to generate/update the property model. A plurality of compounds {Cl} 1<=l<=L may be input to the updated property model and a prediction result list {Rl}j and an ML score Sj may be output or generated. That is, an ML score Sj and further prediction result list {Rl}j may be generated based on the plurality of compounds {Cl} 1<=l<=L input to the updated property model.

In step 506, the prediction result list {Rl}j and the ML score Sj for the current iteration j is received by the RL technique/selection model. In step 508, it is determined whether to retrain the selection model to select a set of compounds for the shortlist of compounds based on the ML score Sj and previous ML score(s) {Sₖ} for 1<=k<j. For example, the property model score {Sₖ} 1<=k<j from a previous iteration (e.g. k=j−1) may be compared with the property model score Sⱼ of the current iteration. If there is an improvement in quality/accuracy in the performance of the property model then this is fed back to the RL technique as a reward and the selection model may be retrained (e.g. ‘Y’). The updated property model may then be retained/kept for another iteration of training the selection model. In step 510, the selection model associated with the RL technique may be updated/retrained based on the reward. The selection model training process 500 proceeds to the next iteration (e.g. j=j+1) and the retrained selection model may then be used in step 502 to select another set of compounds from the result prediction list as the shortlist of compounds for validation.

In step 508, if the comparison between ML scores Sⱼ and previous ML score(s) {Sₖ} for 1<=k<j results in there not being an improvement in quality/accuracy in the performance of the property model in the current iteration, then this is fed back to the RL technique as a penalty and the selection model may be retrained (e.g. ‘Y’). In step 510, the selection model associated with the RL technique may be updated/retrained based on the penalty. Given that the property model has worsened in performance, it may be reverted back to a previously retained/kept property model to before the property model had poor performance. The selection model training process 500 may proceed to the next iteration (e.g. j=j+1) and the retrained selection model may then be used in step 502 to select another set of compounds from the result prediction list as the shortlist of compounds for validation.

In step 508, it may be determined that the selection model is fully trained and that further training does not necessarily improve the selection of the shortlist of compounds. For example, if no improvement can be seen in the predictive property model then the selection model may be considered to be trained and further training may be unnecessary. For example, one method of determining that the selection model is fully trained may include checking whether the selected shortlist of compounds sent for testing in the laboratory and/or by computer simulation do not make any subsequent predictive property model, generated by retraining the ML technique based on the laboratory or computer simulation results, worse and/or the same. Comparing previous property model scores with the current re-trained property model score may be useful in determining whether the selection model can be considered to be fully trained. For example, the selection model may be considered to be trained when comparing the updated property model score with previous retained/kept property model score(s) indicates a plateau of property model scores.

Other modifications to the process 500 may include in response to determining to retrain the selection model in step 510, the updated property model may be reverted to a previous property model when the ML score does not reach a property model performance threshold compared with the corresponding previous ML score. Alternatively or additionally, in step 510, the updated property model may be retained rather than replace by a previously trained property model when the ML score is indicative of meeting or exceeding the property model performance threshold compared with the corresponding previous ML score.

Further modifications may be made that allows the selection model to be trained by the RL technique to not only select a shortlist of compounds but to also select the validation method of using either computer analysis/simulation and/or laboratory experimentation. Given the cost of performing laboratory experimentation, it may be preferable to include a rule that penalises the RL technique when the selection model selects the validation method to be laboratory experimentation too early in the training process or when there are still improvements to be made using computer analysis/simulation.

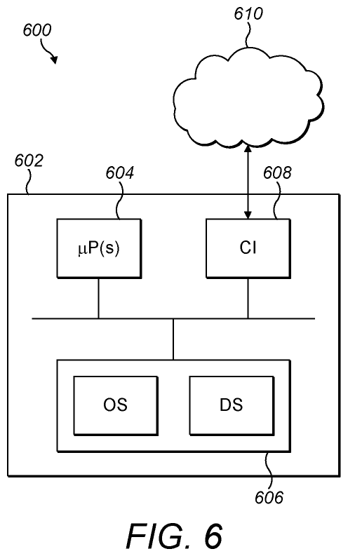

FIG. 6 is a schematic diagram of a computing system 600 comprising a computing apparatus or device 602 according to the invention. The computing apparatus or device 602 may include a processor unit 604, a memory unit 606 and a communication interface 608. The processor unit 604 is connected to the memory unit 606 and the communication interface 608. The memory unit 406 may include an operating system (OS) and a data store (DS) that may include other applications and/or software such as, by way of example only but not limited to, computer-implemented method(s), process(es) and/or instruction code for implementing the method(s) and/or process(es) as described herein with reference to FIGS. 1a to 5. The processor unit 604 and memory 606 may be configured to implement one or more steps of one or more of the process(es) 100, 500 and/or as described herein. The processor unit 604 may include one or more processor(s), controller(s) or any suitable type of hardware(s) for implementing computer executable instructions to control apparatus 602 according to the invention. The computing apparatus 602 may be connected via communication interface 608 to a network 612 for communicating and/or operating with other computing apparatus/system(s) (not shown) for implementing the invention accordingly.

The computing system 600 may be a server system, which may comprise a single server or network of servers configured to implement the invention as described herein. In some examples the functionality of the server may be provided by a network of servers distributed across a geographical area, such as a worldwide distributed network of servers, and a user may be connected to an appropriate one of the network of servers based upon a user location.

Further modifications or examples, may include a computer-implemented method or a method for predicting whether a compound has a particular property using a model (e.g. a property model) trained and/or generated according to any of the process(es) 100, 130, 500 and/or apparatus/systems 120, 300, 400, 600, and/or any method(s)/process(es), modifications thereof, as described with reference to any one or more FIGS. 1a to 6, and/or as herein described and the like. Further modifications or examples, may include a computer-implemented method or a method for generating a property model for predicting whether a compound has a particular property according to any of the process(es) 100, 130, 500 and/or apparatus/systems 120, 300, 400, 600, and/or any method(s)/process(es), modifications thereof, as described with reference to any one or more FIGS. 1a to 6, and/or as herein described and the like.

An apparatus or computing device 602 including a processor 604 (or processor unit), a memory unit 606 and/or a communication interface 608, where the processor 604 may be connected to the memory unit 606 and/or the communication interface 608, where the processor 604, communication interface 608 and/or memory unit 606 are configured to implement the computer-implemented method for using a model (e.g. a property model) to predict whether a compound has a particular property. Alternatively or additionally, the processor 604, communication interface 608 and/or memory unit 606 of the apparatus or computing device 602 may be configured to implement the computer-implemented method for generating or training a property model for predicting whether a compound has a particular property.

Other modifications or examples may include a system for generating a property model based on an ML technique (e.g. an RL technique or any other ML technique), the property model is configured to predict whether a compound is associated with a particular property. The system may include: a model generation module, device or apparatus configured according to any of the process(es) 100, 130, 500 and/or apparatus/systems 120, 300, 400, 600, and/or any method(s)/process(es), step(s) of these process(es), modifications thereof, as described with reference to any one or more FIGS. 1a to 6, the model generation module configured for training a ML technique to generate the property model; a model test module configured for generating a prediction result for a compound and their association with the particular property using the property model, a validation module for validating the property model based on the compound from the prediction result having an association with the particular property, and a model update module for updating the property model based on the property model validation.

The system may include one or more further modifications, features, steps and/or features of the process(es) 100, 130, 500 and/or apparatus/systems 120, 300, 400, 600, computer-implemented method(s) thereof, and/or modifications thereof, as described with reference to any one or more FIGS. 1a to 6, and/or as herein described. For example, the model generation module/device, model test module/device, validation module/device, and/or model update module/device may be configured to implement one or more further modifications, features, steps and/or features of the process(es) 100, 130, 500 and/or apparatus/systems 120, 300, 400, 600, computer-implemented method(s) thereof, and/or modifications thereof, as described with reference to any one or more FIGS. 1a to 6, and/or as herein described.

Furthermore, the process(es) 100, 130, 500 and/or apparatus/systems 120, 300, 400, 600, and/or any method(s)/process(es), step(s) of these process(es), modifications thereof, as described with reference to any one or more FIGS. 1a to 6 may be implemented in hardware and/or software. For example, the method(s) and/or process(es) for training and/or implementing a property model and/or for using a property model described with reference to one or more of FIGS. 1a-6 may be implemented in hardware and/or software such as, by way of example only but not limited to, as a computer-implemented method by one or more processor(s)/processor unit(s) or as the application demands. Such apparatus, system(s), process(es) and/or method(s) may be used to generate an ML model including data representative of a ML model generated from training an ML technique as described with respect to the process(es) 100, 130, 500 and/or apparatus/systems 120, 300, 400, 600, and/or any method(s)/process(es), step(s) of these process(es), as described with reference to any one or more FIGS. 1a to 6, modifications thereof, and/or as described herein and the like. Thus, a ML model or property model may be obtained from apparatus, systems and/or computer-implemented process(es), method(s) as described herein.

Furthermore, a ML selection and/or validation model may also be obtained from the process(es) 100, 130, 500 and/or apparatus/systems 120, 300, 400, 600, and/or any method(s)/process(es), step(s) of these process(es), modifications thereof, as described with reference to any one or more FIGS. 1a to 6, modifications thereof, and/or as described herein, some of which may be implemented in hardware and/or software such as, by way of example only but not limited to, a computer-implemented method that may be executed on a processor or processor unit or as the application demands, as described with reference to one or more of FIGS. 1a-6, modifications thereof, and/or as described herein and the like. In another example, a computer-readable medium that includes data or instruction code representative of a ML model and/or a property model generated based on training a ML technique described with respect to the process(es) 100, 130, 500 and/or apparatus/systems 120, 300, 400, 600, and/or any method(s)/process(es), step(s) of these process(es), as described with reference to any one or more FIGS. 1a to 6, modifications thereof, and/or as described herein and the like, which when executed on a processor, causes the processor to implement the ML model and/or property model.

The above description discusses embodiments of the invention with reference to a single user for clarity. It will be understood that in practice the system may be shared by a plurality of users, and possibly by a very large number of users simultaneously.

The embodiments described above are fully automatic. In some examples a user or operator of the system may manually instruct some steps of the process(es)/method(s) to be carried out.

In the described embodiments of the invention the system may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface). The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages. Variants should be considered to be included into the scope of the invention.

Any reference to ‘an’ item refers to one or more of those items. The term ‘comprising’ is used herein to mean including the method steps or elements identified, but that such steps or elements do not comprise an exclusive list and a method or apparatus may contain additional steps or elements. As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices. Further, as used herein, the term “exemplary” is intended to mean “serving as an illustration or example of something”.

Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A computer-implemented method for generating a property model, the property model for predicting whether a compound is associated with a particular property, the method comprising:
training a machine learning (ML) technique to generate the property model;
generating a prediction result for one or more compounds and their association with the particular property using the property model;
validating the property model based on the one or more compounds from the prediction result having an association with the particular property; and
updating the property model based on the property model validation.

2. A computer-implemented method of claim 1, further comprising: repeating at least the generating and validation steps using the updated property model until determining the property model has been validly trained.

3. A computer-implemented method of claim 1, the method further comprising:
generating a prediction result for a plurality of compounds and their association with the particular property using the property model; and
validating the property model based on the compounds from the prediction result list having an association with the particular property.

4. A computer-implemented method of claim 1, wherein the ML technique is initially trained based on a labelled training dataset associated with a subset of a plurality of compounds in relation to the particular property.

5. A computer-implemented method of claim 1, wherein:
validating the property model further comprises validating a shortlist of compounds from the prediction result list having an association with the particular property; and
updating the property model further comprises updating the property model based on training the ML technique with a labelled training dataset including the validated shortlist of compounds.

6. A computer-implemented method of claim 5, wherein updating the property model further comprising:
generating a further labelled training dataset based on the validated shortlist of compounds and any previously labelled training dataset associated with the particular property; and
retraining the ML technique based on the generated labelled training dataset.

7. A computer-implemented method as claimed in claim 5, wherein validating the shortlist of compounds further comprises:
determining whether to perform laboratory experimentation based on the particular property and the shortlist of compounds; and
in response to determining to perform laboratory experimentation, using experimental results from the laboratory experimentation to estimate the association each compound on the shortlist of compounds has with the particular property.

8. A computer-implemented method as claimed in claim 7, wherein determining to perform laboratory experimentation is based on one or more from the group of:
a number of validation iterations exceeding a validation iteration threshold in which simulation analysis has been consecutively performed for validating the shortlist;
an indication that laboratory analysis will yield an improvement in an ML score for the property model based on previous property model scores calculated from corresponding prediction result lists generated after each shortlist of compounds has been validated; or
a combination on a number of validation iterations and an indication that laboratory experimentation will provide an improved property model.

9. The computer-implemented method according to claim 7, wherein determining whether to perform laboratory experiments further comprises:
determining whether the selected shortlist of compounds has substantially changed from a previously selected shortlist of compounds;
in response to determining that the selected shortlist of compounds has not substantially changed from the previously selected shortlist of compounds, electing to perform laboratory experimentation on a selected subset of compounds from the selected shortlist of compounds.

10. A computer-implemented method as claimed in claim 5, wherein validating the shortlist further comprises:
determining whether to perform simulation analysis based on the particular property and the shortlist of compounds; and
in response to determining to perform simulation analysis, using simulation results from the simulation analysis to estimate the association each compound on the shortlist of compounds has with the particular property.

11. A computer-implemented method as claimed in claim 10, wherein determining to perform simulation analysis is based on one or more from the group of:
a number of validation iterations exceeding a validation iteration threshold in which simulation analysis has been consecutively performed for validating the shortlist;
an indication that simulation analysis will yield an improvement in an ML score for the property model based on previous property model scores calculated from corresponding prediction result lists generated after each shortlist of compounds has been validated; or
a combination on a number of validation iterations and an indication that simulation analysis will provide an improved property model.

12. A computer-implemented method as claimed in claim 10, wherein the number of validation iterations in which simulation analysis is performed consecutively is greater than the number of validation iterations in which laboratory analysis is performed.

13. A computer-implemented method as claimed in claim 12, wherein laboratory analysis is performed once for each of a plurality of generation and validation iterations in which simulation analysis is performed consecutively.

14. The computer-implemented method according to claim 5, wherein the prediction result list comprises a prediction score of whether said each compound has the particular property, the method further comprising selecting the shortlist of compounds from the prediction result list based, at least in part, on the prediction score.

15. A computer-implemented method according to claim 14, wherein validating the shortlist of compounds further comprises selecting one or more compounds for the shortlist of compounds from the prediction result list based on whether a compound has a prediction score indicative of a borderline prediction score.

16. The computer-implemented method according to claim 15, wherein the prediction score comprises a certainty score, wherein compounds that are known to have the particular property are given a positive certainty score, compounds that are known not to have the particular property are given a negative certainty score, and other compounds are given an uncertainty score between the positive certainty score and negative certainty score.

17. The computer-implemented method according to claim 16, wherein the certainty score is a percentage certainty score, wherein the positive certainty score is 100%, the negative certainty score is 0%, and the uncertainty score is between the positive and negative certainty scores.

18. The computer-implemented method according to claim 5, wherein selecting the shortlist of compounds from the prediction result list further comprises selecting one or more compounds having an uncertain prediction result.

19. The computer-implemented method according to claim 5, wherein selecting the shortlist of compounds from the prediction result list further comprises selecting one or more compounds that are dissimilar to the compounds used in any labelled training data used so far.

20. The computer-implemented method according to claim 5, wherein selecting the shortlist of compounds from the prediction result list further comprises using a selection model for selecting the shortlist of compounds from the prediction result list, wherein the selection model is generated by training a reinforcement learning, RL, technique.

21. The computer-implemented method according to claim 20, wherein generating the selection model based on the RL technique further comprising:
selecting, using the selection model, a set of compounds for the shortlist of compounds from the prediction result list for validation;
validating whether the selected shortlist of compounds has the particular property; and
updating the property model based on the ML technique and the validated shortlist of compounds;
generating an ML score and further prediction result list based on the updated property model; and
determining whether to retrain the selection model to select a set of compounds for the shortlist of compounds based on the ML score and previous ML score(s).

22. The computer-implemented method according to claim 21, in response to determining to retrain the selection model, the method further comprising:
reverting the updated property model to a previous property model when the ML score does not reach a property model performance threshold compared with the corresponding previous ML score;
retaining the updated property model to a previously trained property model when the ML score is indicative of meeting or exceeding the property model performance threshold compared with the corresponding previous ML score; and
retraining the selection model to select a set of compounds from the corresponding prediction result list based on the ML score; and
repeating the steps of claim 21 until the selection model is determined to be trained.

23. A computer-implemented method of claim 22, wherein determining the selection model is trained further comprises:
comparing the retained property model score with previous retained property model score(s); and
determining the selection model has been validly trained based on a plateau of property model scores.

24. A computer-implemented method according to claim 5, wherein determining whether the property model has been validly trained further comprises determining the property model has been validly trained based on an indication that further validation of a shortlist is unnecessary.

25. A computer-implemented method according to claim 1, wherein validating the property model further comprising:
generating a property model score based on the prediction result list;
determining whether the property model has been validly trained based on the property model score and previous property model scores.

26. A computer-implemented method of claim 25, wherein determining whether the property model has been validly trained includes determining the property model has been validly trained based on a plateau of property model scores.

27. The computer-implemented method according to claim 1, wherein the ML technique comprises at least one ML technique or combination of ML technique(s) from the group of:
a recurrent neural network configured for predicting, starting from a first compound, a second compound exhibiting a set of desired property(ies);
convolutional neural network configured for predicting, starting from a first compound, a second compound exhibiting a set of desired property(ies);
reinforcement learning algorithm configured for predicting, starting from a first compound, a second compound exhibiting a set of desired property(ies); and
any neural network structure configured for predicting, starting from a first compound, a second compound exhibiting a set of desired property(ies).

28. The computer-implemented method according to claim 1, wherein the particular property includes a property or characteristic indicative of one or more of the following:
a compound docking with another compound to form a stable complex;
a ligand docking with a target protein, wherein the compound is the ligand;
a compound docking or binding with one or more target proteins;
a compound having a particular solubility or range of solubilities;
a compound having a particular toxicity;
any other property or characteristic associated with a compound that can be simulated based on computer simulation(s) and physical movements of atoms and molecules;
any other property or characteristic associated with a compound that can be determined from an expert knowledgebase; and
any other property or characteristic associated with a compound that can be determined from an experimentation.

29. A computer-implemented method according to claim 1, further comprising: further training the property model by iterating over the steps of generating, validating and updating the property model until determining the property model has been validly trained, wherein an updated property model from a previous iteration is used in the generating, validating and updating steps of the current iteration.

30. An apparatus comprising a processor, a memory unit, computer executable instructions, and a communication interface, wherein the processor is connected to the memory unit and the communication interface, wherein the processor and memory are configured to implement the computer-implemented method according to claim 1 when executing the computer executable instructinons.

31. A machine learning model comprising data representative of a ML model generated from training an ML technique according to claim 1.

32. A machine learning model obtained using the computer-implemented method according to claim 1.

33. An apparatus comprising a processor, a memory unit, computer executable instructions, and a communication interface, wherein the processor is connected to the memory unit and the communication interface, wherein the processor and memory are configured to implement a machine learning model comprising data representative of a ML model generated from training an ML technique according to claim 1 when executing the computer executable instructions.

34. A tangible computer-readable medium comprising computer executable instructions representative of a machine learning (ML) model generated based on training a ML technique according to claim 1, which when executed on a processor, causes the processor to implement the ML model.

35. A method for predicting whether a compound has a particular property using a machine learning model trained using the computer-implemented method according to claim 1.

36. A system for generating a property model, the property model for predicting whether a compound is associated with a particular property, the system comprising:
a model generation module for training a machine learning (ML) technique to generate the property model;
a model test module for generating a prediction result for a compound and their association with the particular property using the property model;
a validation module for validating the property model based on the compound from the prediction result having an association with the particular property; and
a model update module for updating the property model based on the property model validation.

37. The system as claimed in claim 36, wherein the model generation module, model test module, validation module, and/or model update module is configured to implement the computer-implemented method according to claim 1.
